(19) United States
(12) Patent Application Publication
(10) Pub. No.: US 2008/0287755 A1
(43) Pub. Date: Nov. 20, 2008

(54) METHOD AND APPARATUS FOR TREND ALERT CALCULATION AND DISPLAY

(75) Inventors: Richard G. Sass, Portland, OR (US); Daniel M. Kaplan, Portland, OR (US); W. Kenneth Ward, Portland, OR (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: ISENSE CORPORATION, Wilsonville, OR (US)

(21) Appl. No.: 12/120,400

(22) Filed: May 14, 2008

Related U.S. Application Data
(60) Provisional application No. 60/938,621, filed on May 17, 2007.

(51) Int. Cl. A61B 5/145

(52) U.S. Cl. 600/309

(57) ABSTRACT

Embodiments of the present invention provide methods, apparatuses, and systems associated with detecting, monitoring, analyzing, and/or displaying analyte level trends in a body. In an embodiment, there is provided a method comprising obtaining a plurality of analyte measurements from a body using an analyte sensing device, and selecting two or more measurements of the plurality of measurements to calculate and display on a device associated with the analyte sensing device a rate of change of the analyte over a period of time spanning the two or more measurements.

### RELATED APPLICATION

The present application is a non-provisional application of U.S. provisional application No. 60/938,621, filed on May 17, 2007, entitled “METHOD AND APPARATUS FOR TREND ALERT CALCULATION AND DISPLAY,” and claims priority to said provisional application. The specification of said provisional application is also hereby fully incorporated by reference in its entirety, except for those sections, if any, that are inconsistent with this specification

### TECHNICAL FIELD

Embodiments of the invention relate generally to the field of medical devices and, specifically, to methods, apparatuses, and systems associated with detecting, monitoring, analyzing, and/or displaying analyte level trends in a body.

### BACKGROUND

In persons with diabetes who take insulin or oral agents, hypoglycemia (low blood sugar) may be a serious event. In some situations, hypoglycemia may lead to loss of cognitive abilities, seizures, stupor or coma. The range of ill effects from hypoglycemia range from embarrassment (losing one's train of thought in a meeting) to more serious outcomes such as auto accidents. For these reasons, detection of hypoglycemia is one of the most important benefits of continuous glucose monitoring. Hyperglycemia (elevated blood sugar) may cause problems as well, such as damage to nerves, blood vessels, and organs, and may lead to further serious conditions such as ketoacidosis or hyperosmolar syndrome.

Thus, it would be beneficial to provide a user with simple and effective mechanisms to monitor the user's glucose values and glucose level trends to aid in the user's management and control of glucose levels. The monitoring, detection, analysis, and/or display of levels or trends for other analytes may provide benefits as well.

### BRIEF DESCRIPTION OF THE DRAWINGS

Embodiments of the present invention will be readily understood by the following detailed description in conjunction with the accompanying drawings. Embodiments of the invention are illustrated by way of example and not by way of limitation in the figures of the accompanying drawings.

FIG. 1 illustrates a display for an electronic monitoring unit in accordance with an embodiment of the present invention;

FIG. 2 illustrates an exemplary electronic monitoring unit showing various display features in accordance with various embodiments of the present invention;

FIG. 3 illustrates an exemplary display element showing varied spacing of rate of change options in accordance with various embodiments of the present invention; and

FIG. 4 illustrates an exemplary analyte sensing system for practicing various embodiments of the present invention.

### DETAILED DESCRIPTION OF EMBODIMENTS OF THE INVENTION

In the following detailed description, reference is made to the accompanying drawings which form a part hereof, and in which are shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means “(A), (B), or (A and B)”. For the purposes of the description, a phrase in the form “at least one of A, B, and C” means “(A), (B), (C), (A and B), (A and C), (B and C), or (A, B and C)”. For the purposes of the description, a phrase in the form “(A)B” means “(B) or (AB)” that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

In various embodiments of the present invention, methods, apparatuses, and systems for detecting, analyzing, and/or displaying analyte level trends in a body are provided. In exemplary embodiments of the present invention, a computing system may be endowed with one or more components of the disclosed apparatuses and/or systems and may be employed to perform one or more methods as disclosed herein.

Embodiments of the present invention provide methods, apparatuses, and systems associated with detecting, monitoring, analyzing, and/or displaying analyte level trends in a body. Embodiments of the present invention provide display mechanisms to indicate analyte level trends in a body.

In an embodiment, a method is provided comprising obtaining a plurality of analyte measurements from a body using an analyte sensing device, and selecting two or more measurements of the plurality of measurements to calculate and display on a device associated with the analyte sensing device a rate of change of the analyte over a period of time spanning the two or more measurements.

In another embodiment, there is provided a method comprising measuring with an analyte sensing device a plurality of analyte values of an individual for a plurality of points of time over a defined time period, determining for the plurality of points of time over the defined time period a rate of change of the plurality of analyte values, and displaying a graphical representation of the rate of change, wherein said determined rate of change is represented as a best fit rate of change from among a plurality of predefined options indicative of relative rates of change, and wherein the best fit rate of change is highlighted.

In embodiments, devices/systems for performing the methods described herein are also provided.

In an embodiment, the phrase “relative rates of change” refers to various rates of change distinguished one from the next by difference in their rates, whether or not the differences are numerically determined.

In an embodiment, the phrase “best fit rate of change” refers to the selected, identified, and/or highlighted rate(s) of change from among a set of predefined options of various rates of change that is/are closest to a calculated/determined rate of change or otherwise satisfies a selection rule directing the selection, identification, display, and/or highlighting of particular predefined options.

In an embodiment, any suitable analyte, such as glucose or lactate, may be measured and associated values may be displayed as described herein.

An embodiment of the present invention provides a mechanism to calculate a rate of change of glucose levels in a body and display that rate of change, or a reasonable substitute, in a graphical form for viewing by a user, a health professional, or other individual. A rate of change may be calculated as the slope of a line between two or more points, or best fit to a plurality of points. Thus, in an embodiment, glucose values may be plotted over time, a line may be fit to those values, and the slope of the line indicating the change in the glucose values over the analyzed time period may be provided. In an embodiment, the slope of the line may be displayed to the user, health professional, or other individual in a variety of ways to provide information about the direction and/or rate of change of the user's glucose values.

In an embodiment, a rate of change (slope) may be determined for 2 or more data values (for example, glucose values) over a period of time. In an embodiment, a suitable period of time may be less than 10 minutes, or, for example, may be from 5-30 minutes or more. Within a defined time period, any suitable number of data values may be obtained, such as 1 per second, 1 per minute, whether regular or irregular in the increments between measurements. In an embodiment, data values may be continuously generated, and two or more data values may be selected representing the analyte values at the selected times, or potentially shifted somewhat in time due to the presence of sensor delay.

In an embodiment, a calculated rate of change may be displayed to a user directly. For example, if the rate of change is calculated to be 2 mg/dl/min, a device may display text that reads “2 mg/dl/min” associated in some manner with an indication that the displayed value represents a rate of change. In an embodiment, a positive rate of change may be shown with or without a “+” sign, and a negative rate of change may be shown with or without a “−” sign, although some indicator that distinguishes positive from negative rates of change, whether textual, symbolic, graphical, colored or otherwise may be used in various embodiments.

In an embodiment, a calculated rate of change may be displayed to a user by displaying a graphic that is representative of the rate of change, whether representing the calculated rate exactly or a best reasonable fit. For example, in an embodiment, a line may be displayed with a slope matching the slope of the line fit to analyte values obtained. In a related embodiment, instead of simply using a line, an arrow may be used pointing in the direction of the slope. In embodiments, other objects or indicators may be used.

In other embodiments, predefined options representing various rates of change may be provided and the particular calculated/determined rate of change may be represented by that option that most closely matches to the calculated/determined rate of change (a “best fit rate of change”), despite the fact that it is not necessarily an exact match. In an embodiment, to avoid understating the extent of a hypoglycemic or hyperglycemic trend, when a calculated/determined rate of change falls between two values, the one that is farthest away from a steady value (no current change) may be selected. Such an embodiment refers to the inclusion of one or more selection rules that direct the selection, identification, display and/or highlighting of a particular option from among a plurality of predefined options. For example, if the selectable options include “0” or “no rate of change,” “2 mg/dl/min,” and “4 mg/dl/min” and a calculated rate of change is 3 mg/dl/min, in an embodiment, the “4 mg/dl/min” option may be selected. A downside of such an approach is that it may increase the number if situations in which a user appears to be in a concerning or dangerous condition (such as a so-called “false alarm”). The alternative approach may be utilized as well (i.e., when a calculated value resides between two defined options, choosing the value closer to “0” or “no change”). Such a system would reduce the number of “false-alarms” but may also under-emphasize the actual state or condition of concern. The above situations illustrate exemplary selection rules, and, in accordance with embodiments of the invention, other rules may be implemented to direct the selection process.

FIG. 1 illustrates an exemplary display for an electronic monitoring unit in accordance with an embodiment of the present invention. FIG. 1 illustrates a current glucose value 102 being displayed in mg/dl in accordance with the selected type of display 104 shown highlighted. Current glucose value 102 is also shown with a direction arrow 106 indicating the current glucose trend for the individual. In addition, the display includes a signal meter 108, a battery power meter 110, and a current date/time indication 112.

In an embodiment, direction arrow 106 may have a slope that matches exactly or substantially matches the slope of the rate of change of glucose values over a defined period of time.

In an alternative embodiment, a display may provide a line, bar, or curve, for example a half circle, such as the right half of a circle, along which may be indicated one or more rates of change. For example, a rate of change may be calculated and a point along the line, bar, or curve representative of the calculated rate of change may be highlighted in some manner. For example, a dot, arrow, or tick mark may appear, illuminate, and/or flash at the location indicative of the calculated rate of change.

For the purposes of various embodiments of the present invention, the term “highlighted” refers broadly to graphically distinguishing one element from another, whether using light, color, tone, brightness, shape, etc.

In embodiments, the identified (for example illuminated) rate of change may be dynamic. In other words, a graphical or textual form may appear that provides a representation that shows the calculated rate of change. In other embodiments, the identified (for example illuminated) rate of change may be one of a predefined group of options each correlating to a defined rate of change. In such an embodiment, the calculated rate of change may be compared to the predefined options to determine the best fit among the predefined options, and that best fit option may be highlighted. In an embodiment, more than one option (object, etc.) may be illuminated, for example, to show that the rate of change falls between the two illuminated predefined options. As described above, in an embodiment, a selection rule may be implemented that provides that the best fit option selected is always the higher or lower option, or the selection of the best fit may be dependent on the particular measurement (a hypoglycemic trend v. hyperglycemic trend, or the steepness/slope of the rate of change).

In an embodiment, a line, bar, or curve may be provided with a plurality of predefined options for the represented rate of change. In other words, a range of options may be predefined, for example “no change,” “+2 mg/dl/min,” “−2 mg/dl/min” etc. In an embodiment, a calculated rate of change may be displayed by highlighting the predefined option that is closest to the calculated rate of change.

In an exemplary embodiment, a curve suggestive of a half clock face from 12 o'clock to 6 o'clock may be used. In such an embodiment, each minute and/or a set thereof (such as every 5th minute, etc.) along the 180 degree sweep (30 minutes) may serve as a predefined option representing a rate of change. In an embodiment, the tick marks, or other graphic, text, and/or number, at each minute or at each defined location may be highlighted in some manner to denote that particular location as representative of the current rate of change of glucose values. In an embodiment, a clock hand may be used, with or without an arrow, to denote the particular minute or defined location along the curve that is representative of the current rate of change of glucose values.

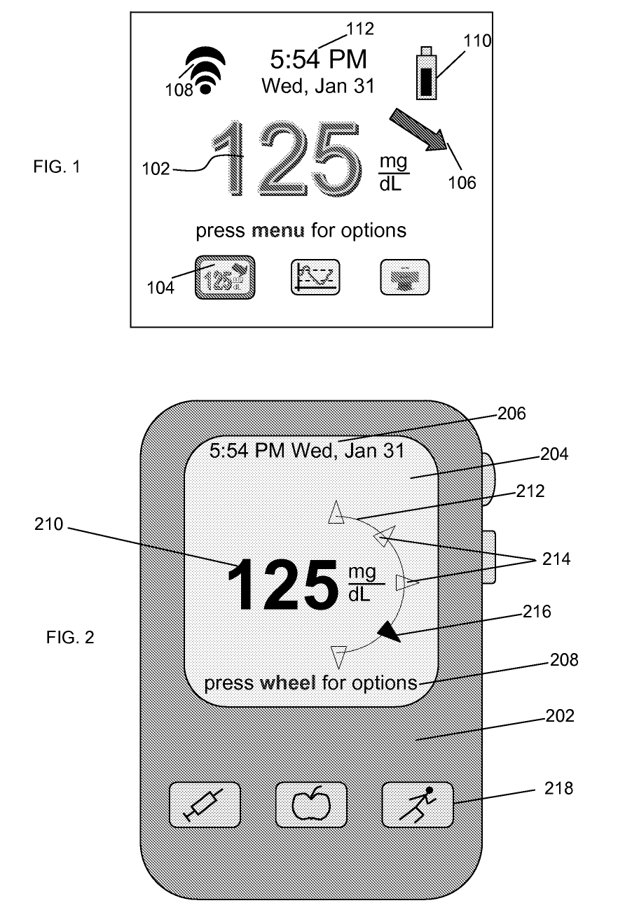

In an embodiment of the present invention as shown in FIG. 2, an exemplary electronic monitoring unit 202 provides various notification and display features. FIG. 2 illustrates a display 204 in accordance with an embodiment of the present invention. Display 204 has a time/date feature 206, which is shown in an exemplary form and, if present, may include the date, time, and/or other elements. In an embodiment, display 204 may also show a variety of text messages, instructions, notifications, etc. such as instruction 208. In an embodiment, display 204 shows a current glucose value 210 being displayed in mg/dl as measured from an individual using the electronic monitoring unit and an associated glucose sensor. In addition, display 204 includes a curve 212, for example a semi-circular curve, with five triangular objects 214. In an embodiment, objects 214 may be utilized to identify the rate of change of the glucose levels/values in the individual. As such, in an embodiment, objects 214 may be predefined to correlate to particular rates of change. As an example, the uppermost object (appearing as an arrow pointing upward) may correlate to a rate of change of glucose of +10 mg/dl/min (or more), whereas the lowermost object (appearing as an arrow pointing downward) may correlate to a rate of change of glucose of −10 mg/dl/min (or more). As a further example, the next objects 214 along the curve may correlate to +/−5 mg/dl/min, and so on.

In an embodiment, opposing or mirror image indicators may have the same or different absolute difference from a “no change” value. As above, in an exemplary situation, opposing indicators may be, for example, +10 mg/dl/min and −10 mg/dl/min. In an alternative embodiment, opposing indicators may be used to indicate the relative levels of concern, which may be different in the hypoglycemic region versus the hyperglycemic region. In such an embodiment, a rate of change of −10 mg/dl/min may be a serious concern and may utilize an arrow or other indicator pointing straight down, while an opposing arrow may be reflective of a rate of change of +15 mg/dl/min to reflect the different levels of concern in the hyperglycemic region, as compared to the hypoglycemic region.

In FIG. 2, while objects 214 are shown as five triangular objects, any number of any suitable shape of objects may be utilized, whether all the same or different. For example, in an embodiment, there may be 3, 5, 7, 9, 11, or more objects or predefined options for glucose trends. Even numbers of objects may be used as well; however, for purposes of symmetry and ease, utilizing an odd number allows for a balanced number of objects around a central object indicative of a steady glucose value (i.e., no change).

In an exemplary embodiment, objects may be provided at 0, +/−18, +/−36, +/−54, +/−72, +/−90 degrees, which may correlate to rates of change of glucose of 0, +/−2, +/−4, +/−6, +/−8, and +/−10 mg/dL/min. Other angles may be used in other embodiments.

In an embodiment, one of objects 214 may be shown highlighted, such as illuminated, colored, flashing, etc. to indicate the current trend, such as illustrated by object 216. In an embodiment, the objects 214 that are not highlighted may be absent or shown in a light shade, colored differently, grayed out, with dashed lines, etc. to ensure that the current trend is clearly distinguishable, for example with highlighted object 216.

In an embodiment, the extent of the increase/decline of glucose may change the manner in which the selected object (such as object 216 in FIG. 2) is highlighted or shown. For example, an object indicating a steady rate of glucose change may show a green object and/or a steady light, whereas an object indicating a moderate rate of glucose change may show a yellow object and/or a pulsing light, and further an object indicating a fast rate of glucose change may show a red object and/or a rapidly beating light. In an embodiment, the highlighted option among the predefined options as mentioned above may also be combined with a display of text stating the current status or trend (such as “slow decline” or “rapid increase” etc.).

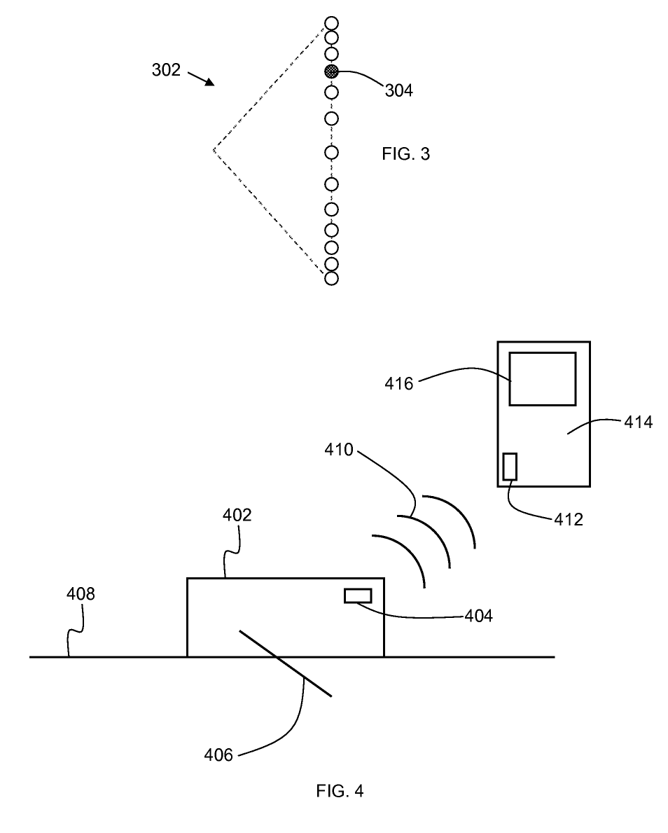

In an embodiment, the options may be equally spaced apart (such as in 2 mg/dl/min increments) or may have varied spacing. For example, in an embodiment, as the rate of change of various values increases (greater positive or negative rates), the spacing between the options may decrease to provide greater accuracy of reporting/display for the user as the level of concern increases. FIG. 3 illustrates an exemplary display element 302 showing varied spacing of rate of change options, with a particular value (rate of change) highlighted (304).

In embodiments, various audible or visual displays of various degrees of concern may be provided by electronic monitoring units, or other associated devices. For example, electronic monitoring unit 202 may provide an indication of an action to be taken based on the condition or degree of concern using various recommendation buttons or lights 218, providing exemplary recommendation options of an injection, a snack (symbolized by an apple), or exercise. An additional recommendation button may, in an embodiment, provide an indication to contact a medical professional.

Embodiments of the present invention may be utilized with a variety of known and later developed glucose sensors or monitors. For example, in an embodiment, the glucose sensor may be a small diameter wire-based device that may be inserted under the skin for 3-7 days. In another embodiment, a suitable sensor may be provided in a device that is fully implantable under the skin and that may remain inserted for 3-12 months. The biosensor(s) may be coupled in various ways to implantable or on-skin electrical components and/or external monitoring units that are capable of performing various calculations and analysis and display of data. In an embodiment, the various graphics described herein may be displayed on the screen of an electronic monitoring unit that may be, for example, worn on a belt or waistband, or in a table-top unit, to which data may be sent by a wired or wireless connection. In an embodiment, the display may provide textual or numeric readouts and/or may show various graphical representations. In an embodiment, the various graphics described herein may be displayed on the patient's personal computer or other computing device.

FIG. 4 illustrates an exemplary analyte sensing system for practicing various embodiments of the present invention. In FIG. 4, an on-skin unit 402 contains various electrical components such as transmitter 404. Extending from and electrically coupled to on-skin unit 402 is analyte sensor 406 which has been inserted into skin 408 of an individual. As analyte sensor 406 obtains analyte values or representative values thereof, and that information may be conveyed to on-skin unit 402 and transmitted (410) by transmitter 404 to a receiver 412 in an electronic monitoring unit 414. Various information regarding the obtained values or calculations associated therewith may be displayed on a display 416 of electronic monitoring unit 414.

In embodiments, various types of notifications, such as alarms or alerts, may be used to indicate the current condition, especially a condition of concern, such as an audible (alarm or electronic voice prompt), visual (for example colored or flashing lights or a symbol on the display), and/or vibratory notification. In an embodiment, a notification may provide an indication of the degree of risk or the condition of concern. In an embodiment, a notification may also provide an indication or suggestion of an action to be taken as a result of the condition of concern.

In an embodiment, for example, if it is determined that there is a moderate risk of hypoglycemia developing in the monitored individual, or if the data indicates a troubling hypoglycemia trend and/or rapid decrease of glucose levels, the sensing system may provide a suggestion to eat a snack in the next 30-60 minutes. In an embodiment, these suggestions may be customized based on the specific medication, exercise, and dietary parameters of an individual. In another example, if an extreme condition of hyperglycemia is identified, there may be provided a notification to contact a health care professional to address the situation.

In an embodiment, either directly from the sensing device or from a separate monitoring unit, a condition of concern may be communicated further to a medical professional as desired or as programmed into the system, whether communicated manually or automatically.

In an embodiment, an audible alarm may be provided to indicate an overt abnormality of glucose, such as a glucose value above or below an established threshold. In an embodiment, an audible alert may be provided to indicate that a rate of change of glucose values is present that, if it continues, may result in an alarm condition. In embodiments, a variety of sounds may be used for alarms and/or alerts. In an embodiment, the sound used for an alarm may be distinguished from that used for an alert. For example, a bell tone may be used for an alert and a chirp or ringing may be used for an alarm. In an embodiment, multiple tones and/or adjustments in volume may be utilized to signify the level of concern.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. A method, comprising:
measuring with an analyte sensing device a plurality of analyte values of an individual for a plurality of points of time over a defined time period;
determining for the plurality of points of time over the defined time period a rate of change of the plurality of analyte values; and
displaying a graphical representation of the rate of change, wherein said determined rate of change is represented as a best fit rate of change from among a plurality of predefined options indicative of relative rates of change, and wherein the best fit rate of change is highlighted.

2. The method of claim 1, wherein said defined time period has a duration of less than 10 minutes.

3. The method of claim 1, wherein said defined time period has a duration of 5 minutes to 30 minutes.

4. The method of claim 1, wherein said graphical representation is displayed on an electronic monitoring unit.

5. The method of claim 1, wherein said graphical representation is displayed on a personal computer.

6. The method of claim 1, wherein said graphical representation depicts a plurality of predefined options indicative of relative rates of change.

7. The method of claim 1, further comprising a secondary notification indicating a current or future condition based on the determined rate of change.

8. The method of claim 7, wherein said secondary notification is displayed as a textual description of the current or future condition.

9. The method of claim 7, wherein said secondary notification comprises an audible alert or alarm.

10. The method of claim 1, further comprising determining the best fit rate of change in accordance with a selection rule incorporating one or more factors to select one rate of change as the best fit of change as opposed to another rate of change.

11. The method of claim 1, wherein highlighting the best fit rate of change comprises highlighting more than one rate of change when the determined rate of change is not an exact match to a single predefined best fit rate of change.

12. A method, comprising:
obtaining a plurality of analyte measurements from a body using an analyte sensing device; and
selecting two or more measurements of the plurality of measurements to calculate and display on a device associated with the analyte sensing device a rate of change of the analyte over a period of time spanning the two or more measurements.

13. The method of claim 12, wherein the displayed rate of change is a representation of the calculated rate of change.

14. The method of claim 12, wherein the displayed rate of change is a best fit representation of the calculated rate of change.

15. The method of claim 12, wherein the rate of change is displayed on the device graphically.

16. The method of claim 12, wherein the rate of change is displayed on the device textually.

17. The method of claim 12, further comprising a secondary notification indicating a current or future condition based on the determined rate of change.

18. The method of claim 17, wherein said secondary notification is displayed as a textual description of the current or future condition.

19. The method of claim 17, wherein said secondary notification comprises an audible alert or alarm.

20. An apparatus, comprising:
an analyte sensing device coupled to an electronic monitoring unit, said electronic monitoring unit comprising a storage medium and a plurality of programming instructions stored in the storage medium adapted to program an apparatus to enable the apparatus to:
measure with an analyte sensing device a plurality of analyte values of an individual for a plurality of points of time over a defined time period;
determine for the plurality of points of time over the defined time period a rate of change of the plurality of analyte values; and
display a graphical representation of the rate of change, wherein said determined rate of change is matched to a best fit rate of change among a plurality of predefined options indicative of relative rates of change, and wherein the best fit rate of change is highlighted.
